(19) United States
(12) Patent Application Publication
(10) Pub. No.: US 2007/0028400 A1
(43) Pub. Date: Feb. 8, 2007

(54) Skin and/or hair treatment agent containing tetrasubstituted cyclohexene compounds

(76) Inventors: Rainer Wolber, Liepmannweg 11, 22399 Hamburg (DE); Cathrin Scherner, (US); Karen Tom Dieck, (US); Jens-Peter Vietzke, (US); Kathrin Schlenz, (US)

Correspondence Address: GREENBLUM & BERNSTEIN, P.L.C., 1950 ROLAND CLARKE PLACE, RESTON, VA, 20191, US

(21) Appl. No.: 10/571,088

(22) PCT Filed: Jul. 10, 2004
(86) PCT No.: PCT/EP04/07627

§ 371 Date: Oct. 4, 2006

(30) Foreign Application Priority Data
Sep. 8, 2003 (DE) 103-41-663.3

(51) Int. Cl. A61K 8/00

(52) U.S. Cl. 8/405; 424/70.1

(57) ABSTRACT

A composition of matter that is suitable for use on skin or hair. The composition comprises one or more compounds of formula 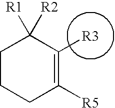 wherein R1, R2, R3 and R5 are as defined in the specification, and at least one other component or element.

The present invention relates to agents for use on the skin and/or the hair, in particular for increasing skin tanning, and also melanin synthesis in the skin or the hair. In particular, the invention relates to cosmetic or dermatological preparations comprising tetrasubstituted cyclohexene compounds. Use of the preparations leads to the induction and intensification of the tanning mechanisms of the skin, to the intensification of the hair color and thus also to an increase in the intrinsic protection of the skin or hair.

The harmful effect of the ultraviolet part of solar radiation on the skin is generally known. While rays with a wavelength of less than 290 nm (the so-called UVC region), are absorbed by the ozone layer in the earth's atmosphere, rays in the range between 290 nm and 320 nm, the so-called UVB region, cause erythema, simple sunburn or even burns of varying severity on the skin.

Numerous compounds are known for protecting against UVB radiation; these are mostly derivatives of 3-benzylidenecamphor, of 4-aminobenzoic acid, of cinnamic acid, of salicylic acid, of benzophenone, and also of 2-phenylbenzimidazole.

It is also important to have available filter substances for the range between about 320 nm and about 400 nm, the so-called UVA region, since its rays too can also cause damage. Thus, it has been found that UVA radiation leads to damage of the elastic and collagenous fibers of connective tissue, causing premature aging of the skin, and that it is to be regarded as a cause of numerous phototoxic and photoallergic reactions. The harmful effect of UVB radiation can be intensified by UVA radiation.

In addition, UVA radiation can cause skin damage by damaging the keratin or elastin within the skin. This leads to a reduction in elasticity and water storage capacity of the skin, i.e. the skin becomes less supple and tends toward wrinkling. This type of wrinkling is also referred to as photoinduced skin aging. The notably high incidence of skin cancer in regions where solar irradiation is strong indicates that damage to the genetic information in cells is also apparently caused by sunlight.

However, UV radiation can also lead to photochemical reactions, the photochemical reaction products intervening in the skin's metabolism.

Such photochemical reaction products are predominantly free-radical compounds, e.g. hydroxyl radicals. Undefined free-radical photoproducts which are formed in the skin itself can also display uncontrolled secondary reactions as a result of their high reactivity. Singlet oxygen, a non-free-radical excited state of the oxygen molecule, can also arise during UV irradiation, as can short-lived epoxides and many others. Singlet oxygen, for example, differs from the normal triplet oxygen (free-radical ground state) by virtue of its increased reactivity. However, excited, reactive (free-radical) triplet states of the oxygen molecules also exist. Such processes are very substantially involved in photoinduced skin aging (inter alia wrinkling) via oxidative damage to various skin structures.

UV radiation is also a type of ionizing radiation. There is therefore the risk that ionic species may also arise during UV exposure, which then, for their part, are capable of oxidative intervention in the biochemical processes.

The pigmentation of the human skin is essentially brought about by the presence of melanin. Melanin and its degradation products (melanoids), carotene, degree of perfusion, and the condition and thickness of the stratum corneum and other skin layers permit skin shades from virtually white (in cases of reduced filling or in cases of an absence of blood vessels) or yellowish via pale brown-reddish, bluish to brown of different shades and ultimately almost black. The individual regions of the skin display differing depths of shade as a result of varying amounts of melanin.

Natural melanin protects the skin from penetrating UV radiation. The number of melanin granules produced in the melanocytes determines whether the person has pale skin or dark skin. In cases of strong pigmentation (e.g. in colored races, but also in those with pale skin following prolonged UV irradiation), melanin is also to be found in the stratum spinosum and even in the stratum corneum. It attenuates the UV radiation by up to about 90% before it reaches the corium.

As characteristic cell organelles, melanocytes contain melanosomes in which the melanin is formed. On excitation by UV radiation, inter alia, melanin is formed to an increased degree. This is transported via the living layers of the epidermis (keratinocytes) ultimately to the horny layer (corneocytes) and causes the more or less marked brownish to brown-black skin color. Melanin is formed as the final stage of an oxidative process in which tyrosine converts, with the assistance of the enzyme tyrosinase, via several intermediates to the brown to brown-black eumelanins (DHICA and DHI melanin) and/or, with participation of sulfur-containing compounds, to the reddish phaeomelanin. DHICA and DHI melanins arise via the common intermediate stages dopaquinone and dopachrome. The latter is converted, partially with participation of further enzymes, either into indole-5,6-quinonecarboxylic acid or into indole-5,6-quinone, from which the two specified eumelanins are formed. The formation of phaeomelanin proceeds, inter alia, via the intermediate products dopaquinone and cysteinyldopa.

Besides various functions of the skin's own melanin, such as, for example, “detoxification”/binding of toxic substances/pharmaceuticals, etc., the function of melanin as a natural UV filter to protect against harmful UV rays, and the antioxidant function of melanin as protection against reactive oxygen species (oxidative stress), which may arise, inter alia, as a result of solar radiation, is very important for the skin. This also with regard to homeostasis, avoidance of skin aging, avoidance of sunburn etc. This thus ought to give rise not only to a cosmetic benefit in the sense of enhanced tanning as a result of the increased synthesis of melanin in the skin following topical application of compounds which increase melanogenesis, but also an additional protection as a result of the various protective powers of melanin.

An object of the present invention is therefore to provide an agent, in particular a cosmetic or dermatological preparation, which intensifies the natural tanning of the skin through increased melanin synthesis and at the same time leads to increased intrinsic protection of the skin.

Depending on their sensitivity to light, the skin types below are normally differentiated:

Skin type I never tans, always burns.

Skin type II rarely tans, burns easily.

Skin type III tans averagely well.

Skin type IV tans easily to give a lasting tan, almost never burns.

Skin type V dark, often almost black skin, never burns.

The natural shielding from harmful UV radiation is a tangible advantage of natural skin tanning. Moreover, for many decades a “healthy” skin color has been a sign of, in particular, sporting activity and is therefore considered to be desirable by a broad section of consumers. Representatives of skin types I and II who wish to enjoy such a skin shade in any case therefore have to rely on self-tanning preparations. However, representatives of skin type III who do not wish to excessively be exposed to the risks of sunbathing but nevertheless want to appear tanned are also thankful target groups for self-tanning preparations.

The simplest way of giving skin a brown shade is to apply appropriately colored make-up preparations. However, only those parts of the body which are covered by the colored preparations are of course colored. With the help of make-up preparations which can be washed off, it is possible to achieve a slight skin tint (e.g. extracts of fresh green walnut shells, henna). A disadvantage of make-up is therefore the time-consuming procedure of application. It is also disadvantageous that they rub off to a great extent onto textiles such as shirt collars or blouses. Moreover, the various dyes can have differing allergenic potential and even have a skin-irritant effect.

It is therefore also an object of the present invention to provide preparations which do not have the disadvantages of make-up tanning preparations.

Artificial tanning can be brought about in a cosmetic or medicinal way, in which case the following approaches essentially play a role:

The regular ingestion of carotene preparations results in carotene being stored in the subcutaneous fatty tissue, and the skin gradually turns orange to yellow-brown.

Coloring can also take place via the route of a chemical change in the horny layer of the skin using so-called self-tanning preparations. The most important active ingredient is dihydroxyacetone (DHA). The skin tanning achieved in this way cannot be washed off and is removed only with the normal desquamation of the skin (after about 10-15 days). Dihydroxyacetone can be referred to as ketotriose and reacts as a reducing sugar with the amino acids in the skin and the free amino and imino groups of keratin via a number of intermediates in the sense of a Maillard reaction to give brown-colored substances, so-called melanoids, which are sometimes also called melanoidins.

A particular disadvantage of tanning with dihydroxyacetone is that the skin tanned with it is not protected against sunburn, in contrast to “sun-tanned” skin.

A further disadvantage of dihydroxyacetone is that, particularly under the influence of ultraviolet radiation, formaldehyde is eliminated, albeit in small amounts in most cases. There was therefore an urgent need to find ways in which the decomposition of dihydroxyacetone can be effectively countered.

An object of the present invention is to find alternatives to DHA as self-tanning agents which do not have disadvantageous properties as are known for DHA.

Coloring by means of self-tanning compositions takes place without exposure to sunlight. In contrast to this, so-called “pre-tan products” or “tan promoters” are also offered, which have to be applied prior to exposure to the sun. In the sun, a yellowing of these preparations then arises, which reportedly leads to a slight brown-yellow coloration of the outer skin, which additionally enhances the “suntan”.

U.S. Pat. No. 5,093,360 describes cosmetic or pharmaceutical preparations which comprise retinal (vitamin A aldehyde) and/or derivatives thereof. Retinal or derivatives thereof are used therein in combination with active agents or as additives in various preparations in order to rectify dermatological disorders. Besides the treatment of acne, mention is also made, inter alia, of tanning preparations which, besides the tanning agents, comprise retinal or derivatives thereof as additive.

There is no indication that retinal or derivatives thereof on their own exert an effect on skin tanning.

A further type of artificial tanning which is likewise completely independent of UV light can be brought about by the hormones which are usually released within the body also as a result of (natural) UV exposure and ultimately stimulate the melanocytes to synthesize melanin. In this connection, mention may be made, for example, of modifications of proopiomelanocortin (POMC), such as aMSH and synthetic variants (such as NDP), some of which have much higher activity than the natural aMSH. Although tanning can in principle be brought about by these hormones, their use in cosmetics is not possible since they are clearly pharmacologically effective substances (hormones) which should not be used widely without medicinal indication.

To overcome the disadvantages of the prior art was likewise the object of the present invention.

In cosmetics, besides skin health and skin care, hair care is also an extremely intensively researched area.

Hair is the thread-like skin appendage consisting of keratin which is virtually universal (lacking on palms of the hand, soles of the feet, extensor sides of the distal phalanges of the toes and fingers); differentiated as long hair (head hair, beard hair, axilla hair, pubic hair=capilli, barba, hirci and pubes, respectively; in men also chest hair), short, bristle hair (supercilia, cilia, vibrissae, tragi) and down (lanugo, vellus hair). The structure of all these hairs is approximately and on the whole similar: in the center the hair medulla (comprising epithelial cells with eosinophilic horny substance granules=trichohyalin granules), surrounded by the hair cortex (comprising keratinized cells; contains pigments) and the outer skin of the hair (cuticula pili; anuclear epidermis layer) and by layers of the epithelial and connective tissue hair sheath.

The hair is divided into the hair shaft protruding from the skin and the inclined hair root reaching into the sub cutis and whose layers correspond approximately to those of the epidermis. The thickened lower root end, the hair bulb, sits on a vascular connective tissue pin, the hair papilla, protruding into it (both as hair base). The bulb in the starting (=anagen) phase of the cyclically repeating hair formation is coated onion-like as a result of the continuous new formation of cells by its near-papillary layer (matrix), then later closed, bulb-like, very keratinized (bulb hair) and is finally, in the end (=telogen) phase, displaced in the direction of the follicle opening by a new hair—starting from a newly forming hair papilla.

Melanin is responsible for personal hair color. The melanin is formed in the melanocytes, cells which arise in the hair bulb associated with the keratinocytes of the hair medulla. Melanocytes contain melanosomes as characteristic cell organelles where the melanin is formed. This is transferred via the long dendrites of the melanocytes to the keratinocytes of the precortical matrix and brings about the more or less marked blond to brown-black hair color. Melanin is formed as the final stage of an oxidative process in which tyrosine is converted, with the assistance of the enzyme tyrosinase, via several intermediates to the brown to brown-black eumelanins (DHICA and DHI melanin) and/or, with participation of sulfur-containing compounds, to the reddish phaeomelanin. DHICA and DHI melanins arise via the common intermediate stages dopaquinone and dopachrome. The latter is converted, partially with participation of further enzymes, either into indole-5,6-quinonecarboxylic acid or into indole-5,6-quinone, from which the two specified eumelanins are formed.

The formation of phaeomelanin proceeds, inter alia, via the intermediate products dopaquinone and cysteinyldopa. Cysteine is additionally necessary when the phaeomelanin is to arise for blond and reddish hair.

The eumelanin is the black-brown pigment. It primarily determines the color depth of the hair. In brown and black hair, it is present in clearly visible granules.

Phaeomelanin is the red pigment. It is responsible for pale blond, blond and red hair. Due to its structure, this melanin is very much finer and smaller. The various proportions of the melanin types lead to the various hair colors:
Blond hair contains a small amount of eumelanin and a large amount of phaeomelanin.
Dark hair contains a large amount of eumelanin and a small amount of phaeomelanin.
Red hair likewise has a small amount of eumelanin and a very large amount of phaeomelanin.
All shades of hair in between result from varying mixing ratios of the two melanin types.

The pigment formation process can only proceed if sufficient tyrosinase is available. This enzyme is formed more infrequently with increasing age. This then gradually leads to gray hair. The reason: with little tyrosinase, less and less tyrosine is also formed. The production of melanin thus decreases. The lack of melanin is replaced by the inclusion of air bubbles. The hair appears gray.

This process is usually slow. It starts at the temples and then extends to the entire head of hair. Subsequently, it affects the beard and the eyebrows. Ultimately, all of the hair on the body is finally gray.

In medicinal terms, gray hair is referred to as canities. There are various graying possibilities. Premature graying, from the age of 20, is also called canities praecox.

Canities symptomatica, or symptomatic graying of the hair, can have various causes. These include:
Pernicious anemia (vitamin B deficiency anemia),
Severe endocrinological disorders, e.g. in the case of thyroid disorders,
Acute febrile illnesses,
Drug side-effects,
Cosmetics,
Metals.

The coloring of hair, in particular of living human hair, using natural dyes, as has been known since antiquity, particularly for the dye henna, and which has been pushed into the background in favor of synthetic dyes has for some years been the object of new interest. A disadvantage is the red shade which arises with henna.

Melanin production, which brings about the hair color, decreases with increasing age: the hair becomes gray or white. It is a cosmetic wish for some consumers to reverse or to slow this process. For this purpose, the cosmetics industry in some countries uses lead acetate which is toxic and therefore prohibited in the European Cosmetics Directive. This lead acetate is preferably applied in the form of a solution to the hair and remains there for a prolonged period without being washed off.

For the dyeing of keratin-containing fibers, e.g. hair, wool or furs, use is generally made either of direct dyes or oxidation dyes, which are formed by oxidative coupling of one or more developer components with one another or with one or more coupler components. Coupler and developer components are also referred to as oxidation dye precursors.

The developer components used are usually primary aromatic amines with a further free or substituted hydroxyl or amino group, situated in the para or ortho position, diaminopyridine derivatives, heterocyclic hydrazones, 4-aminopyrazolone derivatives, and 2,4,5,6-tetraminopyrimidine and derivatives thereof.

Specific representatives are, for example, p-phenylenediamine, p-tolylenediamine, 2,4,5,6-tetraminopyrimidine, p-aminophenol, N,N-bis(2-hydroxyethyl)-p-phenylenediamine, 2-(2,5-diaminophenyl)ethanol, 2-(2,5-diaminophenoxy)ethanol, 1-phenyl-3-carboxyamido-4-amino-5-pyrazolone, 4-amino-3-methylphenol, 2-aminomethyl-4-aminophenol, 2-hydroxymethyl-4-aminophenol, 2-hydroxy-4,5,6-triaminopyrimidine, 2,4-dihydroxy-5,6-diaminopyrimidine and 2,5,6-triamino-4-hydroxypyrimidine.

The coupler components used are usually m-phenylenediamine derivatives, naphthols, resorcinol and resorcinol derivatives, pyrazolones and m-aminophenols. Suitable coupler substances are, in particular, α-naphthol, 1,5-, 2,7- and 1,7-dihydroxynaphthaline, 5-amino-2-methylphenol, m-aminophenol, resorcinol, resorcinol monomethyl ether, m-phenylenediamine, 2,4-diaminophenoxyethanol, 1-phenyl-3-methyl-5-pyrazolone, 2,4-dichloro-3-aminophenol, 1,3-bis(2,4-diaminophenoxy)propane, 2-chlororesorcinol, 4-chlororesorcinol, 2-chloro-6-methyl-3-aminophenol, 2-methylresorcinol and 5-methylresorcinol.

With regard to further customary dye components, reference is made expressly to the series “Dermatology”, published by Ch. Culnan, H. Maibach, Verlag Marcel Dekker Inc., New York, Basle, 1986, Vol. 7, Ch. Zviak, The Science of Hair Care, Ch. 7, pages 248-250 (Direct Dyes), and Ch. 8, pages 264-267 (Oxidation Dyes), and also the “European Inventory of Cosmetic Raw Materials”, 1996, published by the European Commission, obtainable in diskette form from the Bundesverband der deutschen Industrie- und Handelsunternehmen für Arzneimittel, Reformwaren und Körperpflegemittel e.V., Mannheim.

Although intensive colorations with good fastness properties can be achieved with oxidation dyes, the development of the color generally takes place under the influence of oxidizing agents, such as, for example, H₂O₂, which in some cases can result in damage to fibers. Furthermore, some oxidation dye precursors or certain mixtures of oxidation dye precursors can occasionally have a sensitizing effect in people with sensitive skin. Although direct dyes are applied under more moderate conditions, their disadvantage is that the colorations frequently have only inadequate fastness properties.

The object of the present invention is to improve the independent melanin production of hair without having to rely on colorants and, in particular, oxidizing agents such as, for example, H₂O₂. Moreover, the compositions must have no or only a very low sensitizing potential.

Surprisingly, it has now been found that an agent as claimed in claim 1, in particular cosmetic or dermatological preparations as claimed in one of claims 3 to 14, achieves the entire bundle of objects.

The subject-matter of the dependent claims are advantageous embodiments of the agent according to the invention. Furthermore, the invention relates to the use of such agents, and to the compounds according to the invention as agents for increasing skin tanning and/or melanin synthesis in the skin or the hair.

It was surprising and unforeseeable by the person skilled in the art that cosmetic or dermatological preparations, comprising one or more compounds of the structure 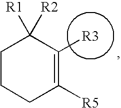
referred to below as tetrasubstituted cyclohexene compounds, achieve the objects.

Where the radicals
R1, R2 and/or R5 are chosen from the group hydrogen, methyl, ethyl, propyl, isopropyl, butyl, tert-butyl, hydroxymethyl, hydroxyethyl, hydroxypropyl, hydroxy and/or carboxylic acid alkyl esters with alkyl radicals chosen from methyl, ethyl, propyl or butyl, preferably methyl;
R3 is chosen from the group of the compound radicals of the structure (I) where 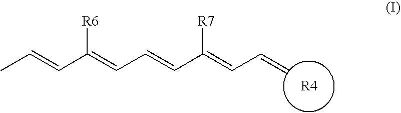
R6 and/or R7 are chosen from the group methyl, hydrogen, ethyl, propyl, isopropyl, butyl, tert-butyl, hydroxymethyl, hydroxyethyl, hydroxypropyl, hydroxy or carboxylic acid alkyl esters where the alkyl radical is chosen from methyl, ethyl, propyl or butyl, preferably methyl;
R4 is chosen from
   carbonyl oxygen,
   amino acid radicals Ala, Ser, Gly, Val, Leu, Ile, Pro, Trp, Phe, Met Tyr, Thr, Cys, Asn, Asp, Glu, Lys, Arg, Gln, H, Orn, Sar, Hyl, Hyp, Hse or Hcy, preferably Ala, Ser or Gly,
   radicals of the structure N—(CH₂)ₓ—OH, N—(CHR9)ₓ—CH2OH, N—(CH₂)ₓ—OCOMe, where in each case x=1-10, N—OH, or
   radicals of the structure 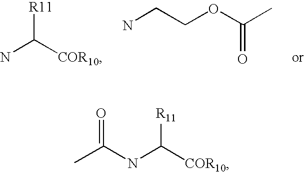
R9 is chosen from hydrogen and/or hydroxy,
R11 is chosen from methyl, hydroxymethyl, hydrogen, prop-2-yl, isobutyl, but-2-yl, pyrrolidine-1,2-diyl, 1H-indol-3-ylmethyl, benzyl; 2-(methylthio)ethyl, 4-hydroxybenzyl, 1-hydroxyethyl, mercaptomethyl, 2-amino-2-oxoethyl, carboxymethyl, carboxyethyl, 4-aminobutyl, 3-{[amino(imino)methyl]-amino}propyl, 3-amino-3-oxopropyl, hydrogen and N-Me, 3-aminopropyl, ethyl, 1H-imidazol-4-ylmethyl, butyl, propyl, 4-amino-3-hydroxybutyl, 4-hydroxypyrrolidine-1,2-diyl, hydroxyethyl, or 2-mercaptoethyl, where if R10=OH then the amino acid radicals are preferably those specified under R4, preferably methyl, hydroxymethyl or hydrogen,
R10 is chosen from hydroxy(—OH), peptidically N-linked amino acid radicals chosen from Ala, Ser, Gly, Val, Leu, Ile, Pro, Trp, Phe, Met Tyr, Thr, Cys, Asn, Asp, Glu, Lys, Arg, Gln, H, Orn, Sar, Hyl, Hyp, Hse or Hcy, preferably Ala, Ser or Gly, radicals of the structure 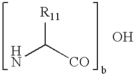
where b=1-6, or 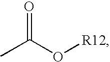
R12 is chosen from mono- to polysaccharides, preferably uniform and/or mixed mono-, di- or trisaccharides, preferably glucose, glycerose, erythrose, threose, ribose, arabinose, lyxose, xylose, allose, altrose, galactose, gulose, idose, mannose or talose.

According to the invention, the compounds 9-retinal of the structural formula 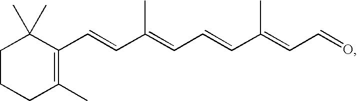
retinal derivatives of the structure 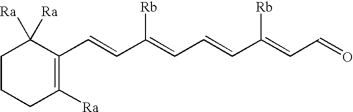
where Ra and/or Rb=alkyl or saturated or unsaturated alkoxy groups and 9-retinal-alkanolamine Schiff's base of the structural formula 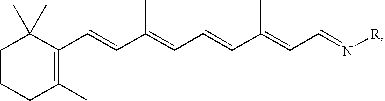
where R=R′(OH)ₙ branched or unbranched alkanols, where R′=C1 to C20 hydrocarbon radical and n=1-5, are excluded from the group of tetrasubstituted cyclohexene compounds according to the invention.

Namely, compounds of the structure (I) with the radicals R1, R2, R5, R6 and R7 as methyl radical or alkyl, saturated or unsaturated alkoxy group and R4 as carbonyl oxygen, and also N—R, where R=R′(OH)ₙ branched or unbranched alkanols, where R′=C1 to C20 hydrocarbon radical and n=1-5, are not covered by the present invention.

It is known that retinal (vitamin A aldehyde) and specific derivatives thereof, as are shown, for example, in U.S. Pat. No. 5,093,360, are supplied in pharmaceutical or cosmetic preparations since they exert a therapeutic effect. Starting from these compounds, which are explicitly excluded from the present invention, there is no inference to the tetrasubstituted cyclohexene compounds according to the invention. It is known that slight changes in the structure of a chemical compound can lead to far-reaching changes in its effect.

Surprisingly, the substances according to the invention, the tetrasubstituted cyclohexene compounds, are exceptionally suitable for bringing about increased skin tanning. All of the compounds of the structures listed above which the person skilled in the art is able to select from the respective groups without exercising inventive activity have been found to be suitable. The person skilled in the art will of course preferably only select those whose compatibility, toxicology or the like are uncritical, especially for the cosmetic or dermatological intended use.

The skin's own melanin has various functions, such as, for example, “detoxification”/binding of toxic substances/pharmaceuticals. Moreover, the function of melanin as natural UV filter to protect against harmful UV rays, and also the antioxidant function of melanin as protection against reactive oxygen species (oxidative stress), which may arise, inter alia, as a result of solar radiation, is very important for the skin. This also with regard to homeostasis, avoidance of skin aging, avoidance of sunburn etc. This thus gives rise not only to a cosmetic benefit in the sense of enhanced tanning as a result of the increased synthesis of melanin in the skin following topical application of compounds, according to the invention, which increase melanogenesis, but also an additional protection as a result of the various protective powers of melanin.

The substances according to the invention are suitable for increasing the physiological tanning of the skin via increased melanin synthesis and thus also for increasing the intrinsic protection of the skin. A significant advantage is that this physiological tanning is achieved without having to expose the skin to natural solar radiation with its harmful effects, or this is required only to a relatively small extent in order to achieve the desired skin tanning. Besides increasing tanning, uneven pigmentations in the skin (“uneven skin tone”) are also evened out. The advantage: the skin appears more uniform, which is desired particularly in the case of aging skin (age spots), melasma and postinflammatory hyperpigmentation.

The invention therefore preferably provides cosmetic or dermatological preparations comprising compounds according to the invention, as defined above. Preparations thus produced are also subject matter of the invention.

In simple terms, the compounds according to the invention comprise cyclic hydrocarbon compounds, where the cyclic structure is preferably constructed from 6 carbon atoms and may be partially to completely unsaturated and additionally has a plurality, in particular 4, hydrocarbon substituents. For simplicity, the compounds according to the invention are referred to as tetrasubstituted cyclohexene compounds. Here, 3 substituents are short-chain, preferably consisting of a methyl group (R1, R2 and R5), a further, fourth substituent (R3), which can consist of a branched and/or partially to completely unsaturated hydrocarbon compound, comprises 1 to 25 C atoms, but preferably at least 4 and at most 20 C atoms. The end of the “fourth substituent” opposite the cyclic structure preferably, but not necessarily, has a polar end.

Through extremely intensive investigations and examinations, it was possible to crystallize out the tetrasubstituted compounds according to the invention as suitable compounds for use on human skin and hair. Of importance here are three basic building blocks, the cyclohexene ring, the structure and chain length of the radical R3, and its functional groups or polarities, respectively. All of these findings lead to the compounds according to the invention, which the person skilled in the art can choose according to the structure (I) and respective radical definitions. The tetrasubstituted cyclohexene compounds according to the invention are known per se, at least from a synthetic point of view, but are not mentioned in the connection and their suitability as agents for use on the skin and the hair. The person skilled in the art can select a compound from the group of compounds listed in claim 1 as required and even combine it with further compounds in order to achieve the effects advantageous according to the invention.

This results in the compound according to the invention having the following general structure: 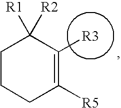
where R1, R2 and R5 is preferably a methyl radical and R3 in the above-mentioned manner is a C1-C25 radical, preferably a C4-C20 radical which preferably has a polar group at the opposite end.

In principle, the topical application of the agents according to the invention in various, in particular W/O and also O/W formulations and other cosmetic application forms is possible and preferred.

Of the compounds according to the invention, N-3,7-dimethyl-9-(2,6,6-trimethylcyclohex-1-en-1-yl)nona-2,4,6,8-tetraen-1-ylidene]alanine is preferred, characterized by the structure 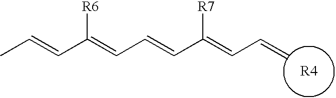
where R4 represents amino acids bonded via an imino functionality, in particular glycine and serine, and R1, R2, R5, R6 and R7 is a methyl radical. In this regard, the following synthesis procedures may be cited by way of example: Rani, Aruna V., Tet Lett, 33, 10, 1992, 1379-1382 and Pshenichnikova, A. B., Russ. J. Bioorg. Chem., 22, 11, 1996, 744-747, and Int J. Vitamin and Nutrition Research, 62, 4, 1992, 298-302 and EP 440078 A2.

N-[(2E,4E,6E,8E)-3,7-dimethyl-9-(2,6,6-trimethylcyclohex-1-en-1-yl)nona-2,4,6,8-tetraen-1-ylidene]-L-alanine 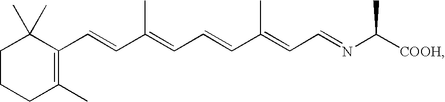
with R1, R2, R5, R6 and R7 as methyl radical and R4 as radical of the structure 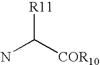
with R11=methyl and R10 chosen as hydroxyl radical.

The compound structure (I) further gives rise to the preferred compounds specified below (IUPAC names) with the given structures:
(1E,2E,4E,6E,8E)-3,7-dimethyl-9-(2,6,6-trimethylcyclohex-1-en-1-yl)nona-2,4,6,8-tetraenal oxime 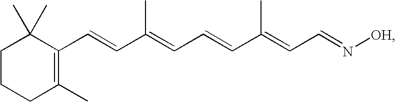
with R1, R2, R5, R6 and R7 as methyl radical and R4 as radical of the structure N—OH.
2-{[(1E,2E,4E,6E,8E)-3,7-dimethyl-9-(2,6,6-trimethylcyclohex-1-en-1-yl)nona-2,4,6,8-tetraen-1-ylidene]amino}ethanol 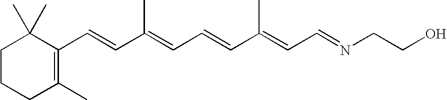
with R1, R2, R5, R6 and R7 as methyl radical and R4 as radical of the structure N—(CH₂)ₓ—OH, where x=2.
1-{[(1E,2E,4E,6E,8E)-3,7-dimethyl-9-(2,6,6-trimethylcyclohex-1-en-1-yl)nona-2,4,6,8-tetraen-1-ylidene]amino}propane-1,2,3-triole 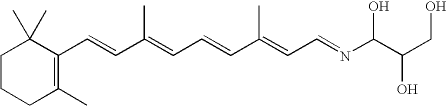
with R1, R2, R5, R6 and R7 as methyl radical and R4 as radical of the structure N—(CHR9)ₓ—CH2OH, where x=2 and R9=OH.
N-[(2E,4E,6E,8E)-3,7-dimethyl-9-(2,6,6-trimethylcyclohex-1-en-1-yl)nona-2,4,6,8-tetraen-1-ylidene]-L-alanyl-L-alanine 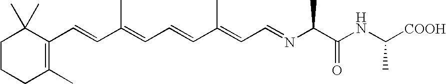
with R4 of the structure 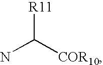
R10 with the structure 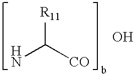
where b=1 and R11=methyl.
N-[(2E,4E,6E,8E)-3,7-dimethyl-9-(2,6,6-trimethylcyclohex-1-en-1-yl)nona-2,4,6,8-tetraen-1-ylidene]-L-seryl-L-serine 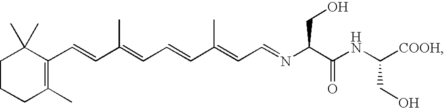
with R4 of the structure 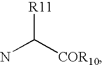
R10 with the structure 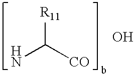
where b=2 and R11=hydroxymethyl.

The compound structure (I) further gives rise to the compound specified below (IUPAC names) with the given structures in a very particularly preferred embodiment:
2-{[(1E,2E,4E,6E,8E)-3,7-dimethyl-9-(2,6,6-trimethylcyclohex-1-en-1-yl)nona-2,4,6,8-tetraen-1-ylidene]amino}ethyl acetate, alternatively also referred to as acetyl-retinyliminoethanol

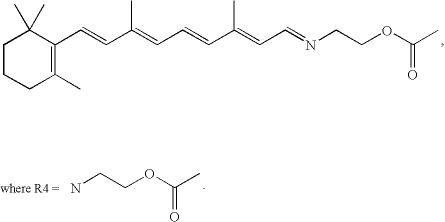

The preparation procedure of acetyl-retinyliminoethanol reads as follows:

Molecular sieve (5 g) and retinal (7) (0.017 mol) are added to a solution of (6) (2.5 g, 0.0204 mol) in abs. ethanol (70 ml). The reaction solution is stirred for 2 days at room temperature.

For work-up, the mixture is filtered and the solvent is removed on a rotary evaporator.

By means of column chromatography (EE/MeOH 9:1), 4.4 g of (8) (70%) are obtained (m.p.: 91-94° C.). Additionally, optionally recrystallization is then carried out (PE/EE).

These compounds according to the invention, where the compounds explicitly specified and shown are only examples of the particular compounds of structure (I), have proven to be useful as agents for use on the skin or the hair. The compounds lead to an increase in melanin synthesis and are preferably to be used as sole additives or as a mixture in cosmetic or dermatological preparations.

Besides the use of the agents as cosmetic or dermatological preparation, a polymer matrix, a skin and/or wound covering, a bandage, a wipe or a pad, a spray, a stick and also textiles, for example bandages or bathing textiles in order to ensure smooth tanning, is also favored as agent according to the invention. An advantage of bandages supplied with the compounds according to the invention is that during the wearing time of the bandage, the skin underneath experiences exactly the same brown coloration as the uncovered skin.

Intensive research has shown that the compounds according to the invention in agents to be applied topically, in particular cosmetic or dermatological preparations, lead to the induction of pigmenting in the skin. Melanogenesis is increased, more melanin forms in the skin, the skin thus becomes browner and the intrinsic protection of the skin is physiologically increased. Also in the case of topical application to hair, the compounds according to the invention in suitable preparations lead to an intensification of the hair color as a result of which natural graying of the hair can also be avoided and even be reversed. Activation of the skin's own tanning and intensification of the hair coloring can take place here of course with or without the involvement of UV light.

To demonstrate the effectiveness of this class of compounds according to the invention, effectiveness tests are carried out in each case. By way of example, the test for the compound acetyl-retinyliminoethanol is given.

A melanogenesis assay was carried out after incubation for 3 days of primary normal human melanocytes with test substance compared to control. The numbers given in the table indicate the melanogenesis rates based on the untreated control (=100%) (measured as C14-tyrosine incorporation). It ensues from this that the melanogenesis, i.e. the process of melanin synthesis, increases to 253% or 117% when the melanocytes are cultivated in the presence of the acetyl-retinyliminoethanol (n=3).

| | Control | 1 μg/ml | 0.1 μg/ml |
|---|---|---|---|
| Average | 100 | 253 | 117 |
| SD | 0 | 77 | 28 |

The compounds according to the invention are characterized inter alia by the fact that—following topical application, in the skin they induce the formation of pigments intrinsic to the skin, increase the synthesis of melanin and in this way produce an enhanced tanning of the skin. They are acceptable in terms of health, non-irritant and easy to handle, and the resulting color shade naturally corresponds to that of the natural healthy skin color. The resulting tan, since it corresponds to the natural tan, is lightfast and cannot be washed off. Surprisingly, the preparations according to the invention also enhance the tanning of skin which is already tanned and, moreover, delay tanned skin from becoming pale.

A further advantage of the present invention arises from the protective properties of natural melanin formed in the skin. Besides various other functions of the melanin intrinsic to the skin (such as, for example, “detoxification” or binding of toxic substances and/or pharmaceuticals etc.), these functions of melanin are also in particular very important for the skin, inter alia with regard to homeostasis, the avoidance of skin aging and the like:

Melanin acts as a natural UV filter for protection against harmful UV rays, and moreover as an antioxidant for protecting against reactive oxygen species (oxidative stress), which can arise, inter alia, as a result of solar irradiation.

Thus, the use according to the invention (i.e. following topical application) results not only in a cosmetic benefit in the sense of enhanced tanning as a result of the increased synthesis of melanin in the skin, but also an additional benefit as a result of the various protective powers of melanin.

The agents according to the invention, e.g. cosmetic or dermatological preparations, induce, in the skin and the hair, the formation of pigments intrinsic to the skin and hair, intensify the existing natural and/or artificial tan of the skin, even out uneven pigmentation of the skin, intensify the natural hair coloration and allow the skin tan and also the hair coloration to last longer.

The formulations according to the invention are entirely satisfactory preparations in every respect which are characterized by a uniformly coloring action. The person skilled in the art could not have foreseen that the formulations according to the invention would
be easier to formulate,
more rapidly and better impart a natural appearance to the skin and the hair,
allow the skin tan and hair coloration to last longer,
have a better effect as moisturizing preparations,
better promote skin smoothing,
be characterized by better care action,
have better sensory properties, such as, for example, spreadability on the skin and the hair, or the ability to be absorbed into the skin, and
offer a better/risk-free intrinsic protection of the skin and hair (against UV radiation) than the preparations of the prior art. In addition, the formulations according to the invention, surprisingly, do not display any hormone effects.

The content of the compounds is between 0.0001 and 30% by weight, advantageously between 0.01 and 10% by weight, particularly advantageously between 0.02 and 2% by weight, in each case based on the total weight of the agent.

The cosmetic and/or dermatological formulations according to the invention can have the customary composition and be used, in particular, for the treatment and care of the skin and/or the hair, as a make-up product in decorative cosmetics or as photoprotective or so-called presun or aftersun preparation. Accordingly, depending on their formula, the formulations according to the invention can be used, for example, as skin protection cream, face cream, cleansing milk, sunscreen lotion, nutrient cream, day or night cream etc.

It is also possible and advantageous for the purposes of the present invention to incorporate the compounds according to the invention into aqueous systems or surfactant preparations for the cleansing and care of the skin and the hair. This includes both shower gels, shampoos, but also conditioners, hair care treatments, hair rinses, hair tonics, sprays etc.

It is of course known to the person skilled in the art that high-quality cosmetic compositions are in most cases inconceivable without customary auxiliaries and additives. These include, for example, builders, fillers, perfume, dyes, emulsifiers, additional active ingredients, such as vitamins or proteins, photoprotective agents, stabilizers, insect repellants, alcohol, water, salts, antimicrobially, proteolytically or keratolytically effective substances etc.

It is also advantageous to administer the compound or compounds according to the invention in encapsulated form, e.g. in collagen matrices and other customary encapsulation materials, such as, for example, cyclic oligosaccharides (in particular alpha-, beta-, HP-beta-, random-Me-beta, gamma-cyclodextrin), where, depending on the chemical properties, known to the person skilled in the art, of the compounds according to the invention, alpha-, beta- or gamma-cyclodextrins are used as encapsulation material. In addition, it may be advantageous to administer the compounds according to the invention or mixtures thereof in the form of cellulose encapsulations, in gelatin, wax matrices or liposomally encapsulated.

In the encapsulation with cyclodextrins, it is assumed that the cyclodextrin backbone acts as the host molecule, and the active ingredient according to the invention acts as the guest molecule. For the preparation, cyclodextrins are dissolved in water, and active ingredient according to the invention is added. The molecular adduct then precipitates out as a solid and can be subjected to customary purification and work-up steps. It is known that cyclodextrin-guest complexes in a corresponding solvent (e.g. water) are in an equilibrium between the concrete guest-cyclodextrin complex and the dissociated form, it being necessary to separate cyclodextrin and guest to a certain degree. Such equilibrium systems are likewise advantageous for the purposes of the present invention.

Corresponding requirements apply mutatis mutandis to the formulation of medicinal preparations.

Medicinal topical compositions for the purposes of the present invention generally comprise one or more medicaments in effective concentration. For the sake of simplicity, for a clear distinction between cosmetic and medicinal application and corresponding products, reference is made to the legal provisions of the Federal Republic of Germany (e.g. Cosmetics Directive, Food and Drugs Act).

In this connection, it is likewise advantageous to add the compound(s) according to the invention as additive to preparations which already comprise other active ingredients for other purposes.

Thus, in the case of the present invention, it has surprisingly been found that the formulations according to the invention are also very particularly suitable for combinations with active ingredients which have a positive effect on the condition of the skin. Thus, it was found that active ingredients for positively influencing aging skin which reduce the formation of wrinkles and also existing wrinkles. Thus in particular in combination with bioquinones, in particular ubiquinone Q10, creatine, creatinine, carnitine, biotin, isoflavone, cardiolipin, lipoic acid, antifreezing proteins, hop and hop-malt extracts. Also promoting agents for restructuring connective tissue, such as isoflavonoids, and isoflavonoid-containing plant extracts, such as, for example, soya and clover extracts, can be used very readily in the formulations according to the invention. It is also found that the formulations are particularly suitable for using active ingredients for assisting skin functions in cases of dry skin, such as, for example, vitamin C, biotin, carnitine, creatine, propionic acid, green tea extracts, eucalyptus oil, urea and mineral salts, such as, for example, NaCl, sea minerals, and osmolytes, such as, for example, taurine, inositol, betaine, quaternary ammonium compounds. In a similar way, the incorporation of active ingredients for alleviating and/or having a positive effect on irritative skin conditions, whether in the case of sensitive skin in general or in the case of skin irritated by noxae (UV light, chemicals) has also proven advantageous. Mention should be made here of active ingredients such as sericosides, various extracts of licorice, licochalcones, in particular licochalocone A, silymarin, silyphos, dexpanthenol, inhibitors of the prostaglandin metabolism, in particular of the cyclooxygenase and of leukotriene metabolism, in particular 5-lipoxygenase, but also of the 5-lipoxygenase inhibitor protein, FLAP. The incorporation of pigmentation modulators has also proven advantageous. Mention is to be made here of active ingredients which reduce skin pigmentation and thus lead to a cosmetically desirable lightening of the skin and/or reduce the appearance of age spots and/or lighten existing age spots. By way of example, mention may be made of tyrosine sulfate, dioic acid (8-hexadecene-1,16-dicarboxylic acid, and lipoic acid and lipoamide, various extracts of licorice. Kojic acid, hydroquinone, arbutin, fruit acids, in particular alpha-hydroxy acids (AHAs), bearberry (Uvae ursi), ursolic acid, ascorbic acid, green tea extracts, aminoguanidine, pyridoxamine. In a similar way, the formulations according to the invention have proven to be excellent combination partners for further active ingredients which bring about enhanced or more rapid tanning of the skin (Advanced Glycation End products (AGE), lipofuscins, nucleic acid oligonucleotides, purins and pyrimidines, NO-releasing substances), be it with or without the effect of UV light.

Particular preference is given to those cosmetic and dermatological preparations which are in the form of a sunscreen. These can advantageously additionally comprise at least one further UVA filter and/or at least one further UVB filter and/or at least one inorganic pigment, preferably an inorganic micropigment.

Surprisingly, cosmetic and dermatological preparations according to the invention are able to bring about an extension of the natural tanning time.

In addition, it was surprising that cosmetic and dermatological formulations according to the invention are able to serve for the treatment of hypopigmentations (vitiligo, uneven pigmentation in aging skin etc.).

According to the invention, the cosmetic and dermatological preparations can comprise cosmetic auxiliaries, as are customarily used in such preparations, e.g. preservatives, bactericides, perfumes, substances for preventing foaming, dyes, pigments which have a coloring effect, thickeners, moisturizing and/or humectant substances, fats, oils, waxes or other customary constituents of a cosmetic or dermatological formulation, such as alcohols, polyols, polymers, foam stabilizers, electrolytes, organic solvents, silicone derivatives or moisturizers.

Moisturizers is the term used to describe substances or mixtures of substances which, following application or distribution on the surface of the skin, confer on cosmetic or dermatological preparations the property of reducing the moisture loss by the horny layer (also called transepidermal water loss (TEWL)) and/or have a beneficial effect on the hydration of the horny layer.

Advantageous moisturizers for the purposes of the present invention are, for example, glycerol, lactic acid, pyrrolidonecarboxylic acid and urea. In addition, it is particularly advantageous to use polymeric moisturizers from the group of polysaccharides which are soluble in water and/or swellable in water and/or gelable in water. Particularly advantageous are, for example, hyaluronic acid and/or a fucose-rich polysaccharide which is listed in Chemical Abstracts under the registry number 178463-23-5 and is available, for example, under the name Fucogel®1000 from SOLABIA S.A.

Glycerol can be used as moisturizer for the purposes of the present application in the range from 0.05-30% by weight, particularly preferably 1-10%.

The amounts of cosmetic or dermatological auxiliaries and carrier substances and perfume to be used in each case can be readily determined depending on the type of product in each case by the person skilled in the art by simple exploratory experiments.

An additional content of antioxidants in the preparations according to the invention is generally preferred. According to the invention, favorable antioxidants which may be used are all antioxidants which are customary or suitable for cosmetic and/or dermatological applications.

It is therefore advantageous to add antioxidants to the preparations according to the invention. The antioxidants are advantageously chosen from the group consisting of amino acids (e.g. glycine, histidine, tyrosine, phenylalanine, tryptophan) and derivatives thereof (in particular N-acetyltyrosine, N-acetylphenylalanine), imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, licopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and also salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisol, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of these specified active ingredients which are suitable according to the invention.

The amount of the abovementioned antioxidants (one or more compounds) in the preparations is preferably 0.001 to 30% by weight, particularly preferably 0.05-20% by weight, in particular 1-10% by weight, based on the total weight of the preparation.

If vitamin E and/or derivatives thereof are the antioxidant or antioxidants, it is advantageous to choose their respective concentrations from the range from 0.001-10% by weight, based on the total weight of the formulation. If vitamin A or vitamin A derivatives, or carotenes or derivatives thereof are the antioxidant or antioxidants, it is advantageous to choose their respective concentrations from the range from 0.001-10% by weight, based on the total weight of the formulation.

Besides one or more oil phases, cosmetic or dermatological formulations for the purposes of the present invention may preferably additionally comprise one or more water phases and be present, for example, in the form of W/O, O/W, W/O/W or O/W/O emulsions. Such emulsions can preferably also be a microemulsion, a Pickering emulsion or a sprayable emulsion.

Moreover, the formulations according to the invention can, however, also advantageously be in the form of oil-free preparations, such as, for example, gels, or in the form of anhydrous preparations.

In addition, the formulations according to the invention can advantageously also comprise dihydroxyacetone or nut extracts, and further substances which are intended to retain or produce or additionally intensify the tan.

The lipid phase of the emulsion according to the invention can advantageously be chosen from the following group of substances:
mineral oils, mineral waxes
oils, such as triglycerides of capric acid or of caprylic acid, but preferably castor oil;
fats, waxes and other natural and synthetic fatty bodies, preferably esters of fatty acids with alcohols of low C number, e.g. with isopropanol, propylene glycol or glycerol, or esters of fatty alcohols with alkanoic acids of low C number or with fatty acids;
alkyl benzoate;
silicone oils, such as dimethylpolysiloxanes, diethylpolysiloxanes, diphenyl-polysiloxanes, and mixed forms thereof.

The oil phase of the emulsions, oleogels or hydrodispersions or lipodispersions for the purposes of the present invention is advantageously chosen from the group of esters of saturated and/or unsaturated, branched and/or unbranched alkanecarboxylic acids with a chain length of from 3 to 30 C atoms and saturated and/or unsaturated, branched and/or unbranched alcohols with a chain length of from 3 to 30 carbon atoms, from the group of esters of aromatic carboxylic acids and saturated and/or unsaturated, branched and/or unbranched alcohols with a chain length of from 3 to 30 C atoms. Such ester oils can then advantageously be chosen from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

In addition, the oil phase can advantageously be chosen from the group of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids with a chain length of from 8 to 24, in particular 12-18, C atoms. The fatty acid triglycerides can, for example, advantageously be chosen from the group of synthetic, semi-synthetic and natural oils, e.g. olive oil, sunflower oil, soybean oil, peanut oil, rapeseed oil, almond oil, palm oil, coconut oil, palm kernel oil and the like.

Any mixtures of such oil and wax components can also be used advantageously for the purposes of the present invention. It may also in some circumstances be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride, dicaprylyl ether.

Mixtures of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate are particularly advantageous.

Of the hydrocarbons, paraffin oil, squalane and squalene are to be used advantageously for the purposes of the present invention.

The oil phase can advantageously also have a content of cyclic or linear silicone oils or consist entirely of such oils, although it is preferred to use an additional content of other oil phase components apart from the silicone oil or the silicone oils.

Cyclomethicone (octamethylcyclotetrasiloxane) is advantageously used as silicone oil to be used according to the invention. However, other silicone oils can also be used advantageously for the purposes of the present invention, for example hexamethylcyclotrisiloxane, polydimethylsiloxane, poly(methylphenylsiloxane).

Mixtures of cyclomethicone and isotridecyl isononanoate, of cyclomethicone and 2-ethylhexyl isostearate are particularly advantageous.

The aqueous phase of the preparations according to the invention optionally advantageously comprises
alcohols, diols or polyols of low C number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol, glycerol, and in particular one or more thickeners which can advantageously be chosen from the group silicon dioxide, aluminum silicates, polysaccharides and derivatives thereof, e.g. hyaluronic acid, xanthan gum, hydroxypropylmethylcellulose, particularly advantageously from the group of polyacrylates, preferably a polyacrylate from the group of so-called Carbopols, for example Carbopol grades 980, 981, 1382, 2984, 5984, in each case individually or in combination.

In addition, UV filter substances may be added to the preparation according to the invention.

Particularly advantageous UV filter substances which are liquid at room temperature for the purposes of the present invention are homomenthyl salicylate (INCI: Homosalate), 2-ethylhexyl 2-cyano-3,3-diphenylacrylate (INCI: Octocrylene), 2-ethylhexyl 2-hydroxybenzoate (2-ethylhexyl salicylate, octyl salicylate, INCI: Octyl Salicylate) and esters of cinnamic acid, preferably 2-ethylhexyl 4-methoxycinnamate (INCI: Octyl Methoxycinnamate) and isopentyl 4-methoxycinnamate (isopentyl 4-methoxycinnamate, INCI: Isoamyl p-Methoxycinnamate).

Preferred inorganic pigments are metal oxides and/or other metal compounds which are insoluble or sparingly soluble in water, in particular oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides, and the sulfate of barium (BaSO₄).

For the purposes of the present invention, the pigment can advantageously also be used in the form of commercially available oily or aqueous predispersions. Dispersion auxiliaries and/or solubilizers may advantageously be added to these predispersions.

According to the invention, the pigments may advantageously be surface-treated (“coated”), the intention being, for example, to form or retain a hydrophilic, amphiphilic or hydrophobic character. This surface treatment can consist in providing the pigments with a thin hydrophilic and/or hydrophobic inorganic and/or organic coating by methods known per se. The various surface coatings according to the present invention may also contain water.

Inorganic surface coatings for the purposes of the present invention may consist of aluminum oxide (Al₂O₃), aluminum hydroxide Al(OH)₃, or aluminum oxide hydrate (also: alumina, CAS No.: 1333-84-2), sodium hexametaphosphate (NaPO₃)₆, sodium metaphosphate (NaPO₃)ₙ, silicon dioxide (SiO₂) (also: silica, CAS No.: 7631-86-9), or iron oxide (Fe₂O₃). These inorganic surface coatings may be present on their own, in combination and/or in combination with organic coating materials.

Organic surface coatings for the purposes of the present invention may consist of vegetable or animal aluminum stearate, vegetable or animal stearic acid, lauric acid, dimethylpolysiloxane (also: dimethicone), methylpolysiloxane (methicone), simethicone (a mixture of dimethylpolysiloxane with an average chain length of from 200 to 350 dimethylsiloxane units and silica gel) or alginic acid. These organic surface coatings may be present on their own, in combination and/or in combination with inorganic coating materials.

Zinc oxide particles and predispersions of zinc oxide particles which are suitable according to the invention are available under the following trade names from the companies listed:

| Trade name | Coating | Manufacturer |
|---|---|---|
| Z-Cote HP1 | 2% Dimethicone | BASF |
| Z-Cote | / | BASF |
| ZnO NDM | 5% Dimethicone | H&R |

Suitable titanium dioxide particles and predispersions of titanium dioxide particles are available under the following trade names from the companies listed:

| Trade name | Coating | Manufacturer |
|---|---|---|
| MT-100TV | Aluminum hydroxide/stearic | Tayca |
| | acid | Corporation |
| MT-100Z | Aluminum hydroxide/stearic | Tayca |
| | acid | Corporation |
| Eusolex T-2000 | Alumina/simethicone | Merck KgaA |
| Titaniumdioxide T805 | Octyltrimethylsilane | Degussa |
| (Uvinul TiO₂) | | |

Advantageous UV-A filter substances for the purposes of the present invention are dibenzoylmethane derivatives, in particular 4-(tert-butyl)-4′-methoxydibenzoylmethane (CAS No. 70356-09-1), which is sold by Givaudan under the trade name Parsol® 1789 and by Merck under the trade name Eusolex® 9020. Advantageous further UV filter substances for the purposes of the present invention are sulfonated, water-soluble UV filters, such as, for example:
phenylene-1,4-bis(2-benzimidazyl)-3,3′-5,5′-tetrasulfonic acid and its salts, particularly the corresponding sodium, potassium or triethanolammonium salts, in particular the phenylene-1,4-bis(2-benzimidazyl)-3,3′-5,5′-tetrasulfonic acid bis-sodium salt with the INCI name Bisimidazylate (CAS No.: 180898-37-7), which is available, for example, under the trade name Neo Heliopan AP from Haarmann & Reimer;
salts of 2-phenylbenzimidazole-5-sulfonic acid, such as its sodium, potassium or its triethanolammonium salt, and the sulfonic acid itself with the INCI name Phenylbenzimidazole Sulfonic Acid (CAS No. 27503-81-7), which is available, for example, under the trade name Eusolex 232 from Merck or under Neo Heliopan Hydro from Haarmann & Reimer;
1,4-di(2-oxo-10-sulfo-3-bornylidenemethyl)benzene (also: 3,3′-(1,4-phenylene-dimethylene)bis(7,7-dimethyl-2-oxobicyclo[2.2.1]hept-1-ylmethanesulfonic acid) and salts thereof (particularly the corresponding 10-sulfato compounds, in particular the corresponding sodium, potassium or triethanolammonium salt), which is also referred to as benzene-1,4-di(2-oxo-3-bornylidenemethyl-10-sulfonic acid). Benzene-1,4-di(2-oxo-3-bornylidenemethyl-10-sulfonic acid) has the INCI name Terephthalidene Dicamphor Sulfonic Acid (CAS No.: 90457-82-2) and is available, for example, under the trade name Mexoryl SX from Chimex;
Sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and salts thereof.

Advantageous UV filter substances for the purposes of the present invention are also so-called broadband filters, i.e. filter substances which absorb both UV-A and also UV-B radiation.

Advantageous broadband filters or UV-B filter substances are, for example, triazine derivatives, such as, for example,
2,4-bis{[4-(2-ethylhexyloxy)-2-hydroxy]phenyl}-6-(4-methoxyphenyl)-1,3,5-triazine (INCI: Aniso Triazine), which is available under the trade name Tinosorb® S from CIBA-Chemikalien GmbH;
diethylhexylbutylamidotriazone (INCI: Diethylhexylbutamidotriazone), which is available under the trade name UVASORB HEB from Sigma 3V;
tris(2-ethylhexyl) 4,4′,4″-(1,3,5-triazine-2,4,6-triyltriimino)trisbenzoate, also: 2,4,6-tris[anilino(p-carbo-2′-ethyl-1′-hexyloxy)]-1,3,5-triazine (INCI: Ethylhexyl Triazone), which is sold by BASF Aktiengesellschaft under the trade name UVINUL® T 150.

An advantageous broadband filter for the purposes of the present invention is also 2,2′-methylenebis(6-(2H-benzotriazole-2-yl)-4-(1,1,3,3-tetramethylbutyl)phenol), which is available under the trade name Tinosorb® M from CIBA-Chemikalien GmbH.

An advantageous broadband filter for the purposes of the present invention is also 2-(2H-benzotriazol-2-yl)-4-methyl-6-[2-methyl-3-[1,3,3,3-tetramethyl-1-[(trimethylsilyl)-oxy]disiloxanyl]propyl]phenol (CAS No.: 155633-54-8) with the INCI name Drometrizole Trisiloxane, which is available under the trade name Mexoryl® XL from Chimex.

The further UV filter substances may be oil-soluble or water-soluble. Advantageous oil-soluble UV-B and/or broadband filter substances for the purposes of the present invention are, for example:
3-benzylidenecamphor derivatives, preferably 3-(4-methylbenzylidene)camphor, 3-benzylidenecamphor;
4-aminobenzoic acid derivatives, preferably 2-ethylhexyl 4-(dimethyl-amino)benzoate, amyl 4-(dimethylamino)benzoate;
derivatives of benzophenone, preferably 2-hydroxy-4-methoxybenzophenone, 2-hydroxy-4-methoxy-4′-methylbenzophenone, 2,2′-dihydroxy-4-methoxy-benzophenone
and UV filters bonded to polymers.
3-(4-(2,2-bisethoxycarbonylvinyl)phenoxy)propenyl)methoxysiloxane/dimethyl-siloxane copolymer which is available, for example, under the trade name Parsol® SLX from Hoffman La Roche.

Advantageous water-soluble filter substances are, for example:

Sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)-sulfonic acid and salts thereof.

A further light protection filter substance to be used advantageously according to the invention is ethylhexyl 2-cyano-3,3-diphenylacrylate (octocrylene), which is available from BASF under the name Uvinul® N 539.

Particularly advantageous preparations for the purposes of the present invention, which are characterized by high or very high UV-A and/or UV-B protection, comprise, besides the filter substance(s) according to the invention, preferably also further UV-A and/or broadband filters, in particular dibenzoylmethane derivatives [for example 4-(tert-butyl)-4′-methoxydibenzoylmethane], phenylene-1,4-bis(2-benzimidazyl)-3,3′-5,5′-tetrasulfonic acid and/or its salts, 1,4-di(2-oxo-10-sulfo-3-bornylidenemethyl)benzene and/or salts thereof and/or 2,4-bis{[4-(2-ethylhexyloxy)-2-hydroxy]phenyl}-6-(4-methoxyphenyl)-1,3,5-triazine, in each case individually or in any combinations with one another.

Also particularly advantageous according to the invention are benzoxazole derivatives, such as, in particular, 2,4-bis[5-1 (dimethylpropyl)benzoxazol-2-yl(4-phenyl)imino]-6-(2-ethylhexyl)imino-1,3,5-triazine with the CAS No. 288254-16-0, which is available, for example, under the trade name Uvasorb® K2A, and hydroxybenzophenones, such as, in particular, hexyl 2-(4′-diethylamino-2′-hydroxybenzoyl)benzoate, and also aminobenzophenone which is available under Uvinul A Plus.

The list of specified UV filters which can be used for the purposes of the present invention is not of course intended to be limiting.

Advantageously, the preparations according to the invention comprise the substances which absorb UV radiation in the UV-A and/or UV-B region in a total amount of, for example, 0.1% by weight to 30% by weight, preferably 0.5 to 20% by weight, in particular 1.0 to 15.0% by weight, in each case based on the total weight of the preparations, in order to provide cosmetic preparations which protect the hair and/or the skin from the entire range of ultraviolet radiation. They can also be used as sunscreens for the hair.

The formulations according to the invention can be used advantageously, but not mandatorily, also in combination with UV radiation—whether artificially produced or natural ultraviolet rays—for example in order to further increase natural tanning or else to achieve a particularly long-lasting tan.

For use, the cosmetic and dermatological formulations according to the invention are applied to the skin and/or the hair in a sufficient amount in the manner customary for cosmetics.

According to the detailed statements, the use of the agent according to the invention, in particular of a cosmetic and/or dermatological preparation, is preferred
as aqueous system and/or surfactant preparation for the cleansing and care of the skin and/or the hair,
as multiple emulsion, microemulsion, Pickering emulsion or sprayable emulsion,
as presun, a sunscreen or aftersun formulation,
for topical application to skin and/or hair,
for tanning the skin,
for caring for the skin,
for the protection of the skin and/or the hair against harmful UV rays,
for increasing the synthesis of melanin in the skin,
for prolonging the brown coloration of the skin,
for protecting the skin against oxidative stress,
for protecting the skin against chronological and light-induced skin aging,
for intensifying the hair color,
for preventing the graying of hair and/or for protecting against the sunlight-induced bleaching of hair,
as shower gel, shampoo, conditioner, hair care treatment, hair rinse, hair tonic, hair spray, make-up, skin protection, face, cleansing, sunscreen, nutrient, day or night cream, gel or lotion or cleansing preparation.

The compounds according to the invention can likewise be a constituent of a polymer matrix, of a skin and/or wound covering, of a bandage, of a wipe or pad, of a spray or applied to or in textiles, such as bandages or bathing textiles.

Thus, incorporation of the compounds into polymer matrices, such as, for example, polyurethane matrices, is possible without problems. Similarly to the known release of active ingredient, the compounds can be released from the matrix onto the skin or the hair where they facilitate their advantageous properties. In a bandage application or applied to textiles, bandages or the like, the compounds are able to penetrate into the skin and bring about the desired protection, care or tanning effect.

Application as spray is preferred since here the compounds only have to be mixed with suitable aerosols or gases.

All amounts, proportions and percentages are based on the weight and the total amount or on the total weight of the preparations, unless stated otherwise.

### EXAMPLES

1. PIT Emulsions

| | 1 | 2 | 3 | 4 | 5 |
|---|---|---|---|---|---|
| Glycerol monostearate self- | 0.50 | | 3.00 | 2.00 | 4.00 |
| emulsifying | | | | | |
| Polyoxyethylene(12) | | 5.00 | | 1.00 | 1.50 |
| cetylstearyl ether | | | | | |
| Polyoxyethylene(20) | | | | 2.00 | |
| cetylstearyl ether | | | | | |
| Polyoxyethylene(30) | 5.00 | | 1.00 | | |
| cetylstearyl ether | | | | | |
| Stearyl alcohol | | | 3.00 | | 0.50 |
| Cetyl alcohol | 2.50 | 1.00 | | 1.50 | |
| 2-Ethylhexyl methoxycinnamate | | | | 5.00 | 8.00 |
| 2,4-bis(4-(2-ethylhexyloxy)-2- | | 1.50 | | 2.00 | 2.50 |
| hydroxy)phenyl)-6-(4- | | | | | |
| methoxyphenyl)-(1,3,5)-triazine | | | | | |
| Butylmethoxydibenzoylmethane | | | 2.00 | | |
| Diethylhexylbutamidotriazone | 1.00 | 2.00 | | 2.00 | |
| Ethylhexyltriazone | 4.00 | | 3.00 | 4.00 | |
| 4-Methylbenzylidenecamphor | | 4.00 | | | 2.00 |
| Octocrylene | | 4.00 | | | 2.50 |
| Phenylene-1,4- | | | 0.50 | | 1.50 |
| bis(monosodium, 2- | | | | | |
| benzimidazyl-5,7-disulfonic | | | | | |
| acid) | | | | | |
| Phenylbenzimidazole sulfonic | 0.50 | | | 3.00 | |
| acid | | | | | |
| C12-15 alkyl benzoate | | 2.50 | | | 5.00 |
| Titanium dioxide | 0.50 | 1.00 | | 3.00 | 2.00 |
| Zinc oxide | 2.00 | | 3.00 | 0.50 | 1.00 |
| Dicaprylyl ether | | | 3.50 | | |
| Butylene glycol | 5.00 | | | 6.00 | |
| dicaprylate/dicaprate | | | | | |
| Dicaprylyl carbonate | | | 6.00 | | 2.00 |
| Dimethicone | | 0.50 | 1.00 | | |
| polydimethylsiloxane | | | | | |
| Phenylmethylpolysiloxane | 2.00 | | | 0.50 | 0.50 |
| Shea butter | | 2.00 | | | 0.50 |
| PVP hexadecene copolymer | 0.50 | | | 0.50 | 1.00 |
| Glycerol | 3.00 | 7.50 | 5.00 | 7.50 | 2.50 |
| Tocopherol acetate | 0.50 | | 0.25 | | 1.00 |
| Acetyl-retinyliminoethanol | 0.15 | | 1.00 | 0.30 | 0.10 |
| Alpha-glucosylrutin | 0.10 | | 0.20 | | |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad | ad | ad | ad | ad |
| | 100 | 100 | 100 | 100 | 100 |

2. O/W Cream

| | Examples | | | | |
|---|---|---|---|---|---|
| | 1 | 2 | 3 | 4 | 5 |
| Glyceryl stearate citrate | | | 2.00 | | 2.00 |
| Glyceryl sterate self-emulsifying | 4.00 | 3.00 | | | |
| PEG-40 stearate | 1.00 | | | | |
| Polyglyceryl-3 methylglucose | | | | 3.00 | |
| distearate | | | | | |
| Licochalcone A | 0.05 | | 0.01 | | |
| Sorbitan stearate | | | | | 2.00 |
| Stearic acid | | 1.00 | | | |
| Polyoxyethylene(20) | | | | | |
| cetylstearyl ether | | | | | |
| Stearyl alcohol | | | 5.00 | | |
| Cetyl alcohol | 3.00 | 2.00 | | 3.00 | |
| Cetylstearyl alcohol | | | | | 2.00 |
| C12-15 Alkylbenzoate | | | | | |
| Caprylic/capric triglyceride | 5.00 | 3.00 | 4.00 | 3.00 | 3.00 |
| Octyldodecanol | | | 2.00 | | 2.00 |
| Dicaprylyl ether | | 4.00 | | 2.00 | 1.00 |
| Paraffinum liquidum | 5.00 | 2.00 | | 3.00 | |
| Titanium dioxide | | | 1.00 | | |
| 4-Methylbenzylidenecamphor | | | 1.00 | | |
| Butylmethoxydibenzoylmethane | | | 0.50 | | |
| Acetyl-retinyliminoethanol | 0.25 | 0.05 | 0.15 | | 0.05 |
| Tocopherol | 0.1 | | | | 0.20 |
| 1-{[(1E,2E,4E,6E,8E)-3,7- | 0.05 | | 0.1 | 0.15 | |
| dimethyl-9-(2,6,6- | | | | | |
| trimethylcyclohex-1-en-1- | | | | | |
| yl)nona-2,4,6,8-tetraen-1- | | | | | |
| ylidene]amino}propane-1,2,3- | | | | | |
| triol | | | | | |
| Biotin | | | 0.05 | | |
| Ethylenediaminetetraacetic acid | 0.1 | | 0.10 | 0.1 | |
| trisodium | | | | | |
| Preservative(s) | q.s. | q.s. | q.s. | q.s. | q.s. |
| Xanthan gum | | | | | |
| Polyacrylic acid | 3.00 | 0.1 | | 0.1 | 0.1 |
| Sodium hydroxide solution 45% | q.s. | q.s. | q.s. | q.s. | q.s. |
| Glycerol | 5.00 | 3.00 | 4.00 | 3.00 | 3.00 |
| Butylene glycol | | 3.00 | | | |
| Perfume | q.s. | q.s. | q.s. | q.s. | q.s. |
| Water | ad | ad | ad | ad | ad |
| | 100 | 100 | 100 | 100 | 100 |

3. O/W Cream

| | Examples | | | | |
|---|---|---|---|---|---|
| | 6 | 7 | 8 | 9 | 10 |
| Glyceryl stearate citrate | | 2.00 | 2.00 | | |
| Glyceryl sterate self-emulsifying | 5.00 | | | | |
| Stearic acid | | | | 2.50 | 3.50 |
| Licochalcone A | | 0.05 | 0.01 | | |
| Stearyl alcohol | 2.00 | | | | |
| Cetyl alcohol | | | | 3.00 | 4.50 |
| Cetylstearyl alcohol | | 3.00 | 1.00 | | 0.50 |
| C12-15 Alkyl benzoate | | 2.00 | 3.00 | | |
| Caprylic/capric triglyceride | 2.00 | | | | |
| Octyldodecanol | 2.00 | 2.00 | | 4.00 | 6.00 |
| N-Acetyltyrosine | 0.5 | | | 0.1 | |
| Paraffinum liquidum | | 4.00 | 2.00 | | |
| Cyclic dimethylpolysiloxane | | | | 0.50 | 2.00 |
| Dimethicone | 2.00 | | | | |
| polydimethylsiloxane | | | | | |
| Titanium dioxide | 2.00 | | | | |
| 1-{[(1E,2E,4E,6E,8E)-3,7- | | 0.10 | | 0.20 | |
| dimethyl-9-(2,6,6- | | | | | |
| trimethylcyclohex-1-en-1- | | | | | |
| yl)nona-2,4,6,8-tetraen-1- | | | | | |
| ylidene]amino}propane-1,2,3- | | | | | |
| triol | | | | | |
| 4-Methylbenzylidenecamphor | 1.00 | | | | 1.00 |
| Butylmethoxydibenzoylmethane | 0.50 | | | | 0.50 |
| Acetyl-retinyliminoethanol | 0.08 | 0.50 | 0.25 | | 0.40 |
| 2,4-Bis(4-(2-ethylhexyloxy)-2- | | 1.0 | 3.0 | | 0.5 |
| hydroxy)phenyl)-6-(4-methoxyphenyl) | | | | | |
| (1,3,5)-triazine | | | | | |
| Dihydroxyacetone | | 0.5 | 2.0 | | 0.5 |
| Tocopherol | | | | | 0.05 |
| Ethylenediaminetetraacetic acid | | | 0.20 | | 0.20 |
| trisodium | | | | | |
| Preservative(s) | q.s. | q.s. | q.s. | q.s. | q.s. |
| Xanthan gum | | | 0.20 | | |
| Polyacrylic acid | 0.15 | 0.1 | | 0.05 | 0.05 |
| Sodium hydroxide solution 45% | q.s. | q.s. | q.s. | q.s. | q.s. |
| Glycerol | 3.00 | | 3.00 | 5.00 | 3.00 |
| Butylene glycol | | 3.00 | | | |
| Ethanol | | 3.00 | | 3.00 | |
| Perfume | q.s. | q.s. | q.s. | q.s. | q.s. |
| Water | ad 100 | ad 100 | ad 100 | ad 100 | ad 100 |

4. W/O Emulsions

| | 1 | 2 | 3 | 4 | 5 |
|---|---|---|---|---|---|
| Cetyldimethicone copolyol | | 2.50 | | 4.00 | |
| Polyglyceryl-2 dipolyhydroxystearate | 5.00 | | | | 4.50 |
| PEG-30 dipolyhydroxystearate | | | 5.00 | | |
| 2-Ethylhexyl methoxycinnamate | | 8.00 | | 5.00 | 4.00 |
| 2,4-Bis(4-(2-ethylhexyloxy)-2- | 2.00 | 2.50 | | 2.00 | 2.50 |
| hydroxy)phenyl)-6-(4- | | | | | |
| methoxyphenyl)(1,3,5)-triazine | | | | | |
| Butylmethoxydibenzoylmethane | | | 2.00 | 1.00 | |
| Diethylhexylbutamidotriazone | 3.00 | 1.00 | | | 3.00 |
| Ethylhexyltriazone | | | 3.00 | 4.00 | |
| 4-Methylbenzylidenecamphor | | 2.00 | | 4.00 | 2.00 |
| Octocrylene | 7.00 | 2.50 | 4.00 | | 2.50 |
| N-Acetyltyrosine | | 0.20 | 0.30 | | |
| Diethylhexylbutamidotriazone | 1.00 | | | 2.00 | |
| Phenylene-1,4- | 1.00 | 2.00 | 0.50 | | |
| bis(monosodium,2-benzimidazyl-5,7-disulfonic | | | | | |
| acid) | | | | | |
| Phenylbenzimidazole sulfonic acid | 0.50 | | | 3.00 | 2.00 |
| Titanium dioxide | | 2.00 | 1.50 | | 3.00 |
| Zinc oxide | 3.00 | 1.00 | 2.00 | 0.50 | |
| Paraffinum liquidum | | | 10.0 | | 8.00 |
| Dihydroxyacetone | | 0.7 | 2.0 | 0.5 | 0.5 |
| C12-15 Alkyl benzoate | | | | 9.00 | |
| Dicaprylyl ether | 10.00 | | | | 7.00 |
| Butylene glycol dicaprylate/ | | | 2.00 | 8.00 | 4.00 |
| dicaprate | | | | | |
| Dicaprylyl carbonate | 5.00 | | 6.00 | | |
| Dimethicone | | 4.00 | 1.00 | 5.00 | |
| polydimethylsiloxane | | | | | |
| Phenylmethylpolysiloxane | 2.00 | 25.00 | | | 2.00 |
| Shea butter | | | 3.00 | | |
| PVP hexadecene copolymer | 0.50 | | | 0.50 | 1.00 |
| Octoxyglycerol | | 0.30 | 1.00 | | 0.50 |
| Glycerol | 3.00 | 7.50 | | 7.50 | 2.50 |
| Glycine soya | | 1.00 | 1.50 | | |
| Magnesium sulfate | 1.00 | 0.50 | | 0.50 | |
| Magnesium chloride | | | 1.00 | | 0.70 |
| Tocopherol acetate | 0.50 | | 0.25 | | 1.00 |
| Acetyl-retinyliminoethanol | 0.15 | 0.08 | 0.5 | 1.00 | 0.80 |
| Preservative | q.s. | q.s. | q.s. | q.s. | q.s. |
| Ethanol | 3.00 | | 1.50 | | 1.00 |
| Perfume | q.s. | q.s. | q.s. | q.s. | q.s. |
| Water | ad 100 | ad 100 | ad 100 | ad 100 | ad 100 |

5. W/O Emulsions

| | 6 | 7 |
|---|---|---|
| Polyglyceryl-2 dipolyhydroxystearate | 4.00 | 5.00 |
| PEG-30 dipolyhydroxystearate | | |
| Lanolin alcohol | 0.50 | 1.50 |
| Isohexadecane | 1.00 | 2.00 |
| Myristyl myristate | 0.50 | 1.50 |
| Vaseline | 1.00 | 2.00 |
| Butylmethoxydibenzoylmethane | 0.50 | 1.50 |
| 4-Methylbenzylidenecamphor | 1.00 | 3.00 |
| Butylene glycol dicaprylate/dicaprate | 4.00 | 5.00 |
| Shea butter | | 0.50 |
| Butylene glycol | | 6.00 |
| Octoxyglycerol | | 3.00 |
| Glycerol | 5.00 | |
| Acetyl-retinyliminoethanol | 0.2 | 0.1 |
| Trisodium EDTA | 0.20 | 0.20 |
| Preservative | q.s. | q.s. |
| Ethanol | | 3.00 |
| Perfume | q.s. | q.s. |
| Water | ad 100 | ad 100 |

6. Hydrodispersions

| | 1 | 2 | 3 | 4 | 5 |
|---|---|---|---|---|---|
| Polyoxyethylene(20) cetylstearyl ether | 1.00 | | | 0.5 | |
| Cetyl alcohol | | | 1.00 | | |
| Sodium polyacrylate | | 0.20 | | 0.30 | |
| Acrylates/C10-30-alkyl acrylate | 0.50 | | 0.40 | 0.10 | 0.10 |
| crosspolymer | | | | | |
| Xanthan gum | | 0.30 | 0.15 | | 0.50 |
| 2-Ethylhexyl methoxycinnamate | | | | 5.00 | 8.00 |
| 2,4-bis(4-(2-ethylhexyloxy)-2- | | 1.50 | | 2.00 | 2.50 |
| hydroxy)phenyl)-6-(4-methoxy- | | | | | |
| phenyl)(1,3,5)-triazine | | | | | |
| Butylmethoxydibenzoylmethane | 1.00 | | 2.00 | | |
| Diethylhexylbutamidotriazone | | 2.00 | | 2.00 | 1.00 |
| Ethylhexyltriazone | 4.00 | | 3.00 | 4.00 | |
| 4-Methylbenzylidenecamphor | 4.00 | 4.00 | | | 2.00 |
| Octocrylene | | 4.00 | 4.00 | | 2.50 |
| Phenylene-1,4-bis(monosodium, | 1.00 | | 0.50 | | 2.00 |
| 2-benzimidazyl-5,7-disulfonic | | | | | |
| acid | | | | | |
| Phenylbenzimidazolesulfonic | 0.50 | | | 3.00 | |
| acid | | | | | |
| Titanium dioxide | 0.50 | | 2.00 | 3.00 | 1.00 |
| Zinc oxide | 0.50 | 1.00 | 3.00 | | 2.00 |
| C12-15 Alkylbenzoate | 2.00 | 2.50 | | | |
| Dicaprylyl ether | | 4.00 | | | |
| Butylene glycol dicaprylate/ | 4.00 | | 2.00 | 6.00 | |
| dicaprate | | | | | |
| Dicaprylyl carbonate | | 2.00 | 6.00 | | |
| Dimethicone | | 0.50 | 1.00 | | |
| polydimethylsiloxane | | | | | |
| Phenylmethylpolysiloxane | 2.00 | | | 0.50 | 2.00 |
| Shea butter | | 2.00 | | | |
| PVP hexadecene copolymer | 0.50 | | | 0.50 | 1.00 |
| Octoxyglycerol | | | 1.00 | | 0.50 |
| Glycerol | 3.00 | 7.50 | | 7.50 | 2.50 |
| Glycine soya | | | 1.50 | | |
| Tocopherol acetate | 0.50 | | 0.25 | | 1.00 |
| Acetyl-retinyliminoethanol | 0.15 | 0.50 | 0.80 | 1.00 | 0.40 |
| Preservative(s) | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad 100 | ad 100 | ad 100 | ad 100 | ad 100 |

7. Gel Cream

| | |
|---|---|
| Acrylate/C10-30 alkyl acrylate | 0.40 |
| crosspolymer | |
| Polyacrylic acid | 0.20 |
| Xanthan gum | 0.10 |
| Cetearyl alcohol | 3.00 |
| C12-15 alkyl benzoate | 4.00 |
| Caprylic/capric triglyceride | 3.00 |
| Cyclic dimethylpolysiloxane | 5.00 |
| Dimethicone polydimethylsiloxane | 1.00 |
| Acetyl-retinyliminoethanol | 0.1 |
| Glycerol | 3.00 |
| Sodium hydroxide | q.s. |
| Preservative | q.s. |
| Perfume | q.s. |
| Water | ad 100.0 |

pH adjusted to 6.0

8. W/O Cream

| | |
|---|---|
| Polyglyceryl-3-diisostearate | 3.50 |
| Glycerol | 3.00 |
| Polyglyceryl-2 dipolyhydroxystearate | 3.50 |
| Acetyl-retinyliminoethanol | 0.25 |
| Preservative(s) | q.s. |
| Perfume | q.s. |
| Water | ad 100.0 |
| Magnesium sulfate | 0.6 |
| Isopropyl stearate | 2.0 |
| Caprylyl ether | 8.0 |
| Cetearyl isononanoate | 6.0 |

9. W/O/W Cream

| | |
|---|---|
| Glyceryl stearate | 3.00 |
| PEG-100 stearate | 0.75 |
| Behenyl alcohol | 2.00 |
| Caprylic/capric triglyceride | 8.0 |
| Octyldodecanol | 5.00 |
| C12-15 Alkyl benzoate | 3.00 |
| Acetyl-retinyliminoethanol | 0.5 |
| Magnesium sulfate (MgSO4) | 0.80 |
| Ethylenediaminetetraacetic acid | 0.10 |
| Preservative(s) | q.s. |
| Perfume | q.s. |
| Water | ad 100.0 |

pH adjusted to 6.0

10. Spray Formulation

| | |
|---|---|
| Ethanol | 28.00 |
| Acetyl-retinyliminoethanol | 0.10 |
| Preservative(s), dyes, perfume | q.s. |
| Propane/butane 25/75 | ad 100 |

11. Shower Bath

| | |
|---|---|
| Sodium laureth sulfate | 33.00 |
| Potassium cocoyl hydrolyzed collagen | 11.00 |
| (30%) | |
| Cocoamphodiacetate (30%) | 5.00 |
| PEG-7 glyceryl cocoate | 2.00 |
| Cocamide MEA | 1.00 |
| Sodium chloride | 0.50 |
| Acetyl-retinyliminoethanol | 0.05 |
| Citric acid | 0.02 |
| Preservative, dyes, perfume | q.s. |
| Water | ad 100 |

12. Hair Treatment

| | |
|---|---|
| Hydroxypropylmethylcellulose | 0.50 |
| Cetrimonium bromide | 1.00 |
| Glycerol | 3.00 |
| Cetearyl alcohol | 2.50 |
| Benzophenone-4 | 0.4 |
| Glyceryl stearate | 2.00 |
| Acetyl-retinyliminoethanol | 0.1 |
| Preservative(s), perfume, pH adjustment | q.s. |
| Water | ad 100 |

The pH is adjusted to 3.5.

13. Hair Rinse

| | |
|---|---|
| Behentrimonium chloride | 1.00 |
| Glycerol | 3.00 |
| Benzophenone-4 | 0.25 |
| Hydroxyethylcellulose | 0.20 |
| Cetearyl alcohol | 3.00 |
| Acetyl-retinyliminoethanol | 0.2 |
| Folic acid | 0.80 |
| Preservative, perfume, pH adjustment | q.s. |
| Water | ad 100 |

The pH is adjusted to 3.0

14. Conditioner Shampoo with Pearlescence

| | 1 | 2 | 3 |
|---|---|---|---|
| Polyquaternium-10 | 0.5 | 0.5 | 0.5 |
| Sodium laureth sulfate | 9.0 | 9.0 | 9.0 |
| Benzophenone-3 | | 0.5 | |
| Benzophenone-4 | | | 0.4 |
| Cocamidopropylbetaine | 2.5 | 2.5 | 2.5 |
| Pearlizing agent | 2.0 | 2.0 | 2.0 |
| Acetyl-retinyliminoethanol | 0.06 | 0.15 | 0.01 |
| Disodium EDTA | 0.1 | 0.2 | 0.15 |
| Preservative(s), perfume, thickener, | q.s. | q.s. | q.s. |
| pH adjustment and solubilizer | | | |
| Water, demineralized | ad 100.0 | ad 100.0 | ad 100.0 |

The pH is adjusted to 6.

15. Clear Conditioner Shampoo

| | 1 | 2 | 3 |
|---|---|---|---|
| Polyquaternium-10 | 0.5 | 0.5 | 0.5 |
| Benzophenone-4 | | 0.4 | |
| 2-Ethylhexyl methoxycinnamate | | | 0.2 |
| Sodium laureth sulfate | 9.0 | 9.0 | 9.0 |
| Cocamidopropylbetaine | 2.5 | 2.5 | 2.5 |
| Acetyl-retinyliminoethanol | 0.02 | 0.05 | 0.05 |
| Iminodisuccinic acid, Na salt | 0.2 | 0.3 | 0.8 |
| Preservative(s), perfume, thickener, | q.s. | q.s. | q.s. |
| pH adjustment and solubilizer | | | |
| Water, demineralized | ad 100.0 | ad 100.0 | ad 100.0 |

The pH is adjusted to 6

16. Clear Light Shampoo with Volume Effect

| | 1 | 2 | 3 |
|---|---|---|---|
| Sodium laureth sulfate | 10.0 | 10.0 | 10.0 |
| Cocamidopropylbetaine | 2.5 | 2.5 | 2.5 |
| Acetyl-retinyliminoethanol | 0.5 | 0.6 | 0.3 |
| Disodium EDTA | 0.2 | 0.15 | 0.7 |
| Preservative(s), perfume, thickener, | q.s. | q.s. | q.s. |
| pH adjustment and solubilizer | | | |
| Water, demineralized | ad 100.0 | ad 100.0 | ad 100.0 |

The pH is adjusted to 5.5

1.-28. (canceled)

29. A composition of matter which is suitable for use on skin or hair, wherein the composition comprises one or more compounds of formula A: 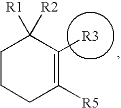
wherein
R1, R2 and R5 are independently selected from hydrogen, methyl, ethyl, propyl, isopropyl, butyl, tert-butyl, hydroxymethyl, hydroxyethyl, hydroxypropyl, hydroxy and carboxylic acid alkyl esters having alkyl radicals selected from methyl, ethyl, propyl and butyl,
R3 is selected from groups of formula (I) 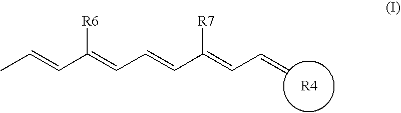
wherein
R6 and R7 are independently selected from hydrogen, methyl, ethyl, propyl, isopropyl, butyl, tert-butyl, hydroxymethyl, hydroxyethyl, hydroxypropyl, hydroxy and carboxylic acid alkyl esters having alkyl radicals selected from methyl, ethyl, propyl and butyl,
R4 is selected from
carbonyl oxygen,
amino acid radicals Ala, Ser, Gly, Val, Leu, Ile, Pro, Trp, Phe, Met Tyr, Thr, Cys, Asn, Asp, Glu, Lys, Arg, Gln, H, Orn, Sar, Hyl, Hyp, Hse or Hcy,
radicals of formulae N—OH, N—(CH₂)ₓ—OH, N—(CHR9)ₓ—CH₂OH, N—(CHR9)ₓ—OH, N—(CH₂)ₓ—OCOMe, where x=1-10, and
radicals of formulae 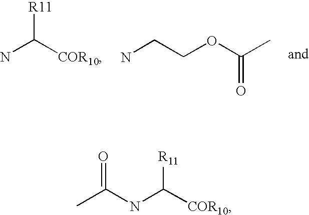
R9 is independently selected from hydrogen and hydroxy,
R 11 is selected from hydrogen, methyl, ethyl, propyl, prop-2-yl, butyl, isobutyl, but-2-yl, hydroxymethyl, 1-hydroxyethyl, pyrrolidine-1,2-diyl, 1H-indol-3-ylmethyl, benzyl, 2-(methylthio)ethyl, 4-hydroxybenzyl, mercaptomethyl, 2-amino-2-oxoethyl, carboxymethyl, carboxyethyl, 4-aminobutyl, 3-{[amino(imino)methyl]-amino}propyl, 3-amino-3-oxopropyl, N-Me, 3-aminopropyl, 1H-imidazol-4-ylmethyl, 4-amino-3-hydroxybutyl, 4-hydroxypyrrolidine-1,2-diyl, hydroxyethyl, and 2-mercaptoethyl,
R10 is selected from hydroxy, peptidically N-linked amino acid radicals selected from Ala, Ser, Gly, Val, Leu, Ile, Pro, Trp, Phe, Met Tyr, Thr, Cys, Asn, Asp, Glu, Lys, Arg, Gln, H, Orn, Sar, Hyl, Hyp, Hse or Hcy, radicals of formula 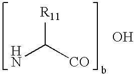
where b=1-6, or 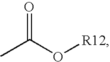
R12 is selected from mono- and polysaccharides;
and at least one additional component or element;
provided that the composition does not comprise one or more of the following compounds as the only compound or compounds of formula A:
(a) 9-retinal of formula 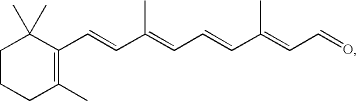
(b) retinal derivatives of formula 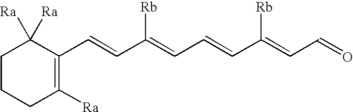
where Ra and Rb are independently selected from alkyl and alkoxy groups; and
(c) 9-retinal alkanolamine Schiff's base of formula 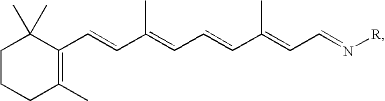
where R=R′(OH)ₙ branched or unbranched alkanols, with R′=C₁₋₂₀ hydro-carbon radical and n=1-5.

30. The composition of matter of claim 29, wherein the composition comprises a cosmetic preparation, a dermatological preparation, a polymer matrix, a skin covering, a wound covering, a bandage, a wipe, a pad, a spray or a textile.

31. The composition of matter of claim 29, wherein the composition is a cosmetic preparation or a dermatological preparation.

32. The composition of matter of claim 30, wherein at least one of R1, R2, R5, R6, and R7 is a methyl radical.

33. The composition of matter of claim 32, wherein R4 is selected from Ala, Ser and Gly.

34. The composition of matter of claim 29, wherein the composition comprises one or more of N-3,7-dimethyl-9-(2,6,6-trimethylcyclohex-1-en-1-yl)nona-2,4,6,8-tetraen-1-ylidene]alanine, (1E,2E,4E,6E,8E)-3,7-dimethyl-9-(2,6,6-trimethylcyclohex-1-en-1-yl)nona-2,4,6,8-tetraenal oxime, 2-{[(1E,2 E,4E,6E,8E)-3,7-dimethyl-9-(2,6,6-trimethyl-cyclohex-1-en-1-yl)nona-2,4,6,8-tetraen-1-ylidene]amino}ethanol, 1-{[(1E,2E,4E,6E,8E)-3,7-dimethyl-9-(2,6,6-trimethylcyclohex-1-en-1-yl)nona-2,4,6,8-tetraen-1-ylidene]amino}propane-1,2,3-triol, N-[(2E,4E,6E,8E)-3,7-dimethyl-9-(2,6,6-trimethylcyclohex-1-en-1-yl)nona-2,4,6,8-tetraen-1-ylidene]-L-alanyl-L-alanine, N-[(2E,4E,6E,8E)-3,7-dimethyl-9-(2,6,6-trimethylcyclohex-1-en-1-yl)nona-2,4,6,8-tetraen-1-ylidene]-L-seryl-L-serine and acetyl-retinyliminoethanol.

35. The composition of matter of claim 29, wherein the composition comprises at least acetyl-retinyliminoethanol as compound of formula A.

36. The composition of matter of claim 29, wherein the composition comprises from 0.0001% to 30% by weight of one or more compounds of formula A.

37. The composition of matter of claim 35, wherein the composition comprises from 0.01% to 10% by weight of one or more compounds of formula A.

38. The composition of matter of claim 32, wherein the composition comprises from 0.02% to 2% by weight of one or more compounds of formula A.

39. The composition of matter of claim 29, wherein the composition comprises one or more compounds of formula A in encapsulated form.

40. The composition of matter of claim 39, wherein an encapsulation material comprises one or more of a collagen matrix, a cyclic oligosaccharide, alpha-, beta-, HP-beta-, random-Me-beta- and gamma-cyclodextrin, cellulose, gelatin, a wax matrix and liposomes.

41. The composition of matter of claim 29, wherein the composition comprises at least one of a UVA filter, a UVB filter and an inorganic pigment.

42. The composition of matter of claim 29, wherein the composition comprises an inorganic micropigment.

43. The composition of matter of claim 29, wherein the composition comprises from 0.001% to 30% by weight of one or more antioxidants.

44. The composition of matter of claim 36, wherein the composition comprises from 0.05% to 20% by weight of one or more antioxidants.

45. The composition of matter of claim 37, wherein the composition comprises from 0.1% to 10% by weight of one or more antioxidants.

46. The composition of matter of claim 29, wherein the composition comprises at least one component selected from preservatives, bactericides, perfumes, substances for preventing foaming, dyes, fillers, pigments which have a coloring effect, thickeners, wetting and/or humectant substances, fats, oils, waxes, alcohols, polyols, polymers, foam stabilizers, electrolytes, organic solvents, silicone derivatives, moisturizers, vitamins, proteins, photoprotective agents, stabilizers, insect repellents, water, salts, antimicrobially, proteolytically or keratolytically effective substances, and medicaments.

47. The composition of matter of claim 29, wherein the composition comprises from 0.05% to 30% by weight of glycerol.

48. The composition of matter of claim 45, wherein the composition comprises from 1% to 10% by weight of glycerol.

49. The composition of matter of claim 29, wherein the composition comprises one or more components selected from active ingredients which have a positive effect on the condition of the skin, promoting agents for restructuring connective tissue, active ingredients for assisting skin functions in cases of dry skin, active ingredients for at least one of alleviating and positively influencing irritated skin states, inhibitors of prostaglandin metabolism, modulators of pigmentation, and active ingredients which bring about an enhanced or more rapid tanning of skin.

50. The composition of matter of claim 49, wherein the composition comprises one or more components selected from bioquinones, creatin, creatinin, carnitine, biotin, isoflavone, cardiolipin, lipoic acid, antifreezing proteins, hop extracts, hop-malt extracts, isoflavonoids, vitamin C, propionic acid, green tea extracts, eucalyptus oil, urea, mineral salts, sea minerals, osmolytes, sericosides, extracts of licorice, licochalcones, silymarin, silyphos, dexpanthenol, inhibitors of cyclooxygenase metabolism, inhibitors of leukotriene metabolism, FLAP, tyrosine sulfate, dioic acid, lipoamide, kojic acid, hydroquinone, arbutin, fruit acids, bearberry (Uvae ursi), ursolic acid, green tea extracts, aminoguanidine, pyridoxamine, Advanced Glycation End products (AGE), lipofuscins, nucleic acid oligonucleotides, purins, pyrimidines, and NO-releasing substances.

51. The composition of matter of claim 29, wherein the composition comprises at least one of an aqueous system and a surfactant preparation which is suitable for cleansing or care of at least one of skin and hair.

52. The composition of matter of claim 29, wherein the composition comprises at least one of a multiple emulsion, a microemulsion, a Pickering emulsion and a sprayable emulsion.

53. The composition of matter of claim 29, wherein the composition comprises at least one of a presun formulation, a sunscreen formulation and an aftersun formulation.

54. The composition of matter of claim 29, wherein the composition is in a form which is suitable for topical application to at least one of skin and hair.

55. A composition for application to at least one of skin and hair, wherein the composition is selected from at least one of a shower gel, a shampoo, a conditioner, a hair care treatment, a hair rinse, a hair tonic, a hair spray, a make-up composition, a skin protection cream, a face cream, a cleansing cream, a sunscreen cream, a nutrient cream, a day cream, a night cream, a gel, a lotion, and a cleansing preparation and comprises the composition of matter of claim 29.

56. A method of tanning skin, wherein the method comprises applying the composition of matter of claim 29 to skin.

57. A method of caring for skin, wherein the method comprises applying the composition of matter of claim 29 to skin.

58. A method of protecting skin or hair from harmful UV rays, wherein the method comprises applying the composition of matter of claim 29 to the skin or hair to be protected.

59. A method of increasing synthesis of melanin in skin, wherein the method comprises applying the composition of matter of claim 29 to skin.

60. A method of prolonging brown coloration of skin, wherein the method comprises applying the composition of matter of claim 29 to skin.

61. A method of protecting skin against oxidative stress, wherein the method comprises applying the composition of matter of claim 29 to skin.

62. A method of protecting skin against chronological and photo-induced skin aging, wherein the method comprises applying the composition of matter of claim 29 to skin.

63. A method of intensifying hair color, wherein the method comprises applying the composition of matter of claim 29 to hair.

64. A method of preventing at least one of graying of hair and for protecting hair against sunlight-induced bleaching, wherein the method comprises applying the composition of matter of claim 29 to hair.

65. The composition of matter of claim 29, wherein the composition is capable of at least one of increasing skin tanning and melanin synthesis in at least one of skin and hair.
